(19) United States
(12) Patent Application Publication
(10) Pub. No.: US 2016/0000687 A1
(43) Pub. Date: Jan. 7, 2016

(54) Cosmetic Composition Forming Anisotropic Microstructures For Reduction Of The Appearance Of Fine Lines And Wrinkles On The Skin

(71) Applicant: ELC Management LLC, Melville, NY (US)

(72) Inventors: Jack Polonka, Peekskill, NY (US); Phillip Cummins, Livingston, NJ (US)

(21) Appl. No.: 14/791,814

(22) Filed: Jul. 6, 2015

Related U.S. Application Data
(60) Provisional application No. 62/021,391, filed on Jul. 7, 2014.

(51) Int. Cl. A61K 8/81; A61Q 7/02; A61Q 19/08; A61Q 19/04; A61Q 19/02; A61Q 17/04

(52) U.S. Cl. CPC A61K 8/8141; A61Q 19/02; A61Q 17/04; A61Q 19/08; A61Q 19/04; A61Q 7/02; A61K 2800/48; A61K 2800/43

(57) ABSTRACT

The present invention provides a cosmetic composition for reducing the appearance of fine lines and wrinkles in skin, comprising a tensor agent and a dermatologically acceptable medium, the tensor agent being present from about 20% to about 80% of the composition, and wherein the tensor agent forms an anisotropic microstructure upon drying on the skin.

### FIELD OF THE INVENTION

The present invention relates to a cosmetic composition capable of diminishing the appearance of fine lines and wrinkles by forming an anisotropic microstructure on a substrate, such as human skin.

### BACKGROUND OF THE INVENTION

Visible wrinkles, particularly those on the face and around the eyes, are among the most prevalent and undesirable signs of aging. Many consumer products and procedures are devoted to hiding or reducing wrinkles. These products and procedures can be simple and inexpensive, for example, applying make-up, particularly a primer or colored foundation, may simply cover the wrinkles on a consumer's skin. Far more expensive and drastic procedures, such as surgical face lifts and Botox injections are also used to reduce the appearance of wrinkles on the face. There are a plethora of lotions and creams which purport to hydrate the skin, making it more supple, and reducing the appearance of wrinkles. Some of these liquid products contain active ingredients that help repair and rejuvenate skin over time. But all of these products and procedures have drawbacks.

Foundation and other make-up products are often visible, offer minimal texture benefits, and have no lasting effect on the skin. Once the make-up is removed, the skin has the same appearance as before the make-up was applied. Liquid products can have chronic, acute, or both effects on the skin. Hydration and optical effects are common acute benefits, and these benefits wear-off over time. Chronic actives may rejuvenate or repair the skin over time. These chronic benefits take time to occur and are incremental improvements. There are limits to how effective these chronic benefits can be. Plastic surgery and injections of chemicals have a more pronounced, immediate and dramatic effect on the look of a consumer's skin, but these procedures can be very expensive and come with many risks. Plastic surgery has the same risk of failure as any other surgical procedure, including disfigurement and infection.

Attempts have been made to develop new categories of products to improve the appearance of skin without the drawbacks of existing products and procedures. One such family of products can be generally classified as “adhesive, contractile film formers”. Film formers are chemical compositions that when applied to skin, leave a pliable, cohesive and continuous covering. A select group of film formers are also adhesive to skin and even contractile. Wrinkles, in their simplest form, are crevices or valleys in the skin. When an adhesive, contractile film former is applied, the skin at the bottom of the valley or crevice may be pulled to the surface, causing skin look smooth and wrinkle-free. The drawbacks of existing adhesive, contractile film forming products include discomfort caused by the contraction of the skin, irritation of the skin, cracking of the film as the consumer uses her face muscles, incompatibility with other cosmetic products in her regimen, uneven distribution of the benefit agent, and visibility of the film which is often whitish and noticeable. Curing or reducing one of these problems has, in the past, exacerbated one of the other problems.

### SUMMARY OF THE INVENTION

The present invention provides a cosmetic composition for reducing the appearance of fine lines and wrinkles in skin, comprising a tensor agent and a dermatologically acceptable medium, the tensor agent being present from about 20% to about 80% of the composition, and wherein the tensor agent forms an anisotropic microstructure upon drying on the skin.

Further forms of the present invention will be appreciated in the detailed description that follows.

### DETAILED DESCRIPTION OF THE INVENTION

All percentages and ratios used herein are by weight of the total composition and all measurements made are at 25° C., unless otherwise designated. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

The term “anisotropic microstructure” as used herein, means a fine formation of rigid or elastic structures which are non-uniform and are directionally dependent. The aniosotropic structures measure differently in terms of physical structure and applied force when measured along different axes.

The terms “apply” or “application” as used in reference to a composition, means to contact or spread the compositions of the present invention onto a substrate such as the human skin surface or epidermis.

The term “dermatologically acceptable” as used herein means that the compositions or components described are suitable for use in contact with human skin tissue without undue toxicity, incompatibility, instability, allergic response, and the like.

The term “facial skin surface” as used herein refers to one or more of forehead, periorbital, cheek, perioral, chin, and nose skin surfaces. While facial skin surfaces are of concern and are exemplified herein, other skin surfaces may be treated with the compositions of the present invention, for example, surfaces typically not covered by clothing such as facial skin surfaces, hand and arm skin surfaces, foot and leg skin surfaces, and neck and chest skin surfaces (e.g., décolletage).

The term “safe and effective amount” as used herein means an amount of a compound or composition sufficient to induce a positive cosmetic benefit.

The terms “stable” and “stability” as used herein mean a composition which is substantially unaltered in chemical state, physical homogeneity and/or color when the composition is at a temperature of from about 1° C. to about 40° C.

The term “tensor agent” as used herein means a polymer which creates a tension force across a substrate, such as human skin, and thereby provides a skin benefit, such as reduction of the appearance of fine lines and wrinkles.

### Cosmetic Composition

The present invention relates to compositions for application to a skin surface. The compositions may be in a wide variety of product forms that include, but are not limited to, solutions, suspensions, lotions, creams, gels, toners, sticks, pencil, sprays, aerosols, ointments, cleansing liquid washes and solid bars, pastes, foams, powders, mousses, wipes, strips, patches, hydrogels, film-forming products, facial and skin masks (with and without insoluble sheet), make-up such as foundations, eye liners, and eye shadows, and the like. The composition form may follow from the particular dermatologically acceptable carrier chosen, if present in the composition.

### Tensor Agent

The cosmetic compositions comprise from about 20% to about 80%, preferably from about 30% to about 80%, and most preferably from about 40% to about 80% of a tensor agent.

The tensor agent herein is a polyurethane/polyester copolymer which may optionally have semicrystalline properties.

The term, “semicrystalline polyester”, as used herein, is understood to mean a polyester having a chain or crystallizable chain parts which have a melting temperature which causes a change of phase of the polymer (solid-liquid transition).

The term “crystallizable chain or block” is understood to mean, for the purposes of the invention, a chain or block which, if it were alone, would pass from the amorphous state to the crystalline state, irreversibly, depending on whether the temperature is above or below this melting temperature.

The term “block” is generally understood to mean a grouping of atoms belonging to the backbone, a grouping constituting one of the repeating units of the polymer. A block generally comprises at least 5 identical repeating units. In one embodiement, a crystallizable block(s) is (are) therefore of a chemical nature which is different from the amorphous block (s). When the crystallizable part is in the form of a crystallizable block of the polymeric backbone, the amorphous part of the polymer is in the form of an amorphous block; the semicrystalline polymer is in this case a block copolymer, for example of the diblock, triblock or multiblock type, containing at least one crystallizable block and at least one amorphous block.

The expression “chain” is understood to mean, for the purposes of the invention, a pendent or side grouping of atoms relative to the polymer backbone. Advantageously, the “crystallizable pendent chain” may be a chain containing at least 6 carbon atoms. When the polymers of the invention are polymers with crystallizable side chains, they are advantageously in a random form.

In one embodiment, the semicrystalline polyurethane/polyester copolymers which can be used in the composition according to the invention have a melting point greater than or equal to 20° C., in particular ranging from 25° C. to 100° C., preferably ranging from 30° C. to 80° C. and even better from 35° C. to 70° C.

The melting point may be measured by any known method and in particular with the aid of a differential scanning calorimeter (D. S. C), for example the calorimeter sold under the name DSC 30 by the company METLER, according to the protocol as described below. A sample of 5 to 10 mg of copolymer placed in a crucible is subjected to a first rise in temperature ranging from −20° C. to 110° C., at the heating rate of 5° C./minute, and is then cooled from 110° C. to −20° C. at a cooling rate of 5° C./minute and finally subjected to a second rise in temperature ranging from −20° C. to 110° C. at a heating rate of 5° C./minute. During the second rise in temperature, the variation of the difference between the power absorbed by the empty crucible and the crucible containing the copolymer sample is measured as a function of time.

The value of the melting point corresponds to the peak maximum of the thermal profile thus obtained.

The semicrystalline copolymer(s) used according to the invention have a number-average molecular mass Mn greater than or equal to 1000.

In one embodiment, the semicrystalline copolymer (s) of the composition of the invention have a number-average molecular mass Mn ranging from 2000 to 800 000, preferably from 3000 to 500 000, even better from 4000 to 150 000, in particular less than 100 000, and even better from 4,000 to 99,000.

In one embodiment, the crystallizable block (s) or chain (s) of the copolymers according to the invention represent at least 20% of the total weight of the copolymer and even better at least 30%.

The polyurethane/polyester copolymers which can be used in the composition according to the invention are generally of synthetic origin.

The composition according to the invention may comprise those semicrystalline polyurethane/polycaprolactone copolymers marketed under the references Tecoflex® CLA-60D-V and Tecoflex® CLA-93A-V resin by Noveon, or those marketed by Merquinsa under the references Pearlbond® and Disperbond®, in particular Disperbond® D31S and Disperbond® D39S, and mixtures thereof. Such polymers are described in U.S. Patent Publication No. 2010/0266520.

When applied to skin, under conditions specified herein, the tensor agent forms an anisotripic microstructure on human skin. The anisotropic nature of the film which forms provides a tension force which is not uniform and generally concentrated along a single axis. When applied to skin at sites of fine lines and/or wrinkles, the unidirectional tension force has a smoothing effect, thereby diminishing the appearance of fine lines and/or wrinkles.

It has been found that in order to effectively lift facial skin, the composition must be capable of applying at least 3 to 4 Newtons of force across the skin's surface.

### Skin Conditioning Agent

It has been found that formation of anisotropic microstructures, which provide an enhanced tension force across skin, are readily formed in the compositions herein when levels of skin conditioning agents are carefully balanced.

Skin conditioning agents may be selected from humectants and emollients and may generally be present at a level of less than about 10% of the composition. In the particular case of humectants, they are preferably present at a level of from about 0% to about 8%, more preferably from about 2% to about 6%, and most preferably at about 5% of the composition. Emollients may be present at a level of less than about 10%, preferably less than about 5%, and most preferably, less than about 3% of the composition. In one embodiment, the compositions are free of humectants and emollients.

Humectants are polyhydric alcohols intended for moisturizing, reducing scaling and stimulating removal of built-up scale from the skin. Typical polyhydric alcohols include polyalkylene glycols and more preferably alkylene polyols and their derivatives. Illustrative examples include propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. When present, the humectant is most preferably hexylene glycol.

When the conditioning agent is an emollient, it may be selected from hydrocarbons, fatty acids, fatty alcohols and esters.

### Plasticizer

It has further been found that formation of anisotropic microstructures, which provide an enhanced tension force across skin, are most readily formed in the compositions herein when level of placticizers is limited. Plasticizers are materials which increase the plasticity or fluidity of a composition.

When present, the placticizer may be incorporated at a level of less than about 5%, more preferably less than 3%, and most preferably less than about 2% of the composition. The plasticizer can be, for example, an alkyl mono-glycol or di-glycol containing 3 to 5 carbon atoms. The plasticizers herein are selected from the group consisting of polyhydric alcohols, water soluble alkoxylated nonionic polymers, and mixtures thereof. Polyhydric alcohols useful herein include glycerin, propylene glycol, 1,3-butylene glycol, 1,3 propanediol, dipropylene glycol, diglycerin, sodium hyaluronate, polypropanediol and mixtures thereof. A commonly utilized plasticizer is propylene glycol. In one embodiment, the compositions are free of plasticizers.

Exemplary plasticizers include: glycerin available from Asahi Denka; propylene glycol with tradename Lexol® PG-865/855 available from Inolex, 1,2-PROPYLENE GLYCOL USP® available from BASF; 1,3-butylene glycol available from Kyowa Hakko Kogyo; dipropylene glycol with the same tradename available from BASF; 1,3 propane diol with tradename ZEMEA® from DuPont Company; polypropanediol with tradename CERENOL® H250 from DuPont® Company diglycerin with tradename DIGLYCEROL® available from Solvay GmbH; sodium hyaluronate with tradename ACTIMOIST® available from Active Organics, AVIAN SODIUM HYALURONATE series available from Intergen, HYALURONIC ACID Na® available from Ichimaru Pharcos.

### Dermatologically Acceptable Carrier

The compositions of the present invention also comprise a dermatologically acceptable carrier. The phrase “dermatologically acceptable carrier”, as used herein, means that the carrier is suitable for topical application to the keratinous tissue, has good aesthetic properties, is compatible with the actives in the composition, and will not cause any unreasonable safety or toxicity concerns. In one embodiment, the carrier is present at a level of from about 20% to about 80%, about 30% to about 70%, about 40% to about 60%, or, alternatively, from about 80% to about 95%, by weight of the composition.

The carrier can be in a wide variety of forms. Non-limiting examples include simple solutions (e.g., aqueous, organic solvent, or oil based), emulsions, suspensions, and solid forms (e.g., gels, sticks, flowable solids, or amorphous materials). In certain embodiments, the dermatologically acceptable carrier is in the form of an emulsion or suspension. Emulsions or suspensions may be generally classified as having a continuous aqueous phase (e.g., oil-in-water and water-in-oil-in-water) or a continuous oil phase (e.g., water-in-oil and oil-in-water-in-oil). The oil phase of the present invention may comprise silicone oils, non-silicone oils such as hydrocarbon oils, esters, ethers, and the like, and mixtures thereof.

Emulsions may further comprise an emulsifier. The composition may comprise any suitable percentage of emulsifier to sufficiently emulsify the carrier. Suitable weight ranges include from about 0.1% to about 10% or about 0.2% to about 5% of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986). Suitable emulsions may have a wide range of viscosities, depending on the desired product form.

The carrier may further comprise a thickening agent as are well known in the art to provide compositions having a suitable viscosity and rheological character.

### Pigments and Powders

The compositions of the present invention can comprise from about 5% to about 45%, preferably from about 5% to about 30% of a pigment powder component. The pigments included in the pigment powder component herein may be hydrophobic in nature, or hydrophobically treated. By keeping the level of pigment component low, the entire composition maintains flexibility to accommodate other components which provide spreadability, moisturization, and fresh and light feel. The species and levels of the pigments are selected to provide, for example, shade, coverage, good wear performance, and stability in the composition.

Pigments useful for the pigment component herein are inorganic and organic powder such as talc, mica, sericite, synthetic fluorphlogopite, pearl pigments such as alumina, barium sulfate, calcium secondary phosphate, calcium carbonate, coverage titanium oxide, finely divided titanium oxide, zirconium oxide, normal particle size zinc oxide, hydroxy apatite, iron oxide, iron titanate, ultramarine blue, Prussian blue, chromium oxide, chromium hydroxide, cobalt oxide, cobalt titanate, titanium oxide coated mica; organic powder such as polyester, polyethylene, polystyrene, methyl methacrylate resin, cellulose, 12-nylon, 6-nylon, styrene-acrylic acid copolymers, polypropylene, vinyl chloride polymer, tetrafluoroethylene polymer, boron nitride, fish scale guanine, laked tar color dyes, and laked natural color dyes. Such pigments may be treated with a hydrophobical treatment agent, including: silicone such as methicone, dimethicone, and perfluoroalkylsilane; fatty material such as stearic acid and disodium hydrogenated glutamate; metal soap such as aluminum dimyristate; aluminum hydrogenated tallow glutamate, hydrogenated lecithin, lauroyl lysine, aluminum salt of perfluoroalkyl phosphate, and aluminum hydroxide as to reduce the activity for titanium dioxide, and mixtures thereof. Such pigments may also be coated with substances considered more hydrophilic such as polysaccharides, caprylyl silane, or polyethylene oxide silane treatments.

Commercially available pigment powder component includes coverage titanium dioxide, such as SI-T-CR-50Z®, SI-Titanium Dioxide IS®, SA-Titanium Dioxide® CR-50®, SI-FTL-300® and SA/NAI-TR-10®, all of them are available from Miyoshi Kasei, iron oxide and cyclopentasiloxane and dimethicone and disodium hydrogenated glutamate: SA/NAI-Y-10/D5(70%)/SA/NAI-R-10/D5(65%)/SA/NAI-B-10/D5(75%) available from Miyoshi Kasei, iron oxide and disodium hydrogenated glutamate: SA/NAI-Y-10/SA/NAI-R-10/SA/NAI-B-10 available from Miyoshi Kasei, iron oxide and methicone: SI Mapico Yellow Light Lemon XLO/SI Pure Red Iron Oxide R-1599/SI Pure Red Iron Oxide R-3098/SI Pure Red Iron Oxide R-4098/SI Black Iron Oxide No.247 available from Daito Kasei, alumina and titanium dioxide and methicone: SI-LTSG30AFLAKE® H (5%) LHC available from Miyoshi Kasei, talc and methicone: SI-Talc JA13R LHC® available from Miyoshi Kasei, mica and methicone: SI Mica available from Miyoshi Kasei, dimethicone: SA-SB-300® available from Miyoshi Kasei, mica and methicone.

Sericite available from Miyoshi Kasei, mica and dimethicone: SA Sericite available from Miyoshi Kasei, mica and C9-15 Fluoroalcol Phosphates and Triethoxy Caprylylsilane: FOTS-52 Sericite FSE available from Daito Kasei, Talc and C9-15 Fluoroalcol Phosphates and triethoxy caprylylsilane: FOTS-52 Talc JA-13R available from Daito Kasei, boron nitride and methicone: SIO2 Boron Nitride SHP-6 available from Daito Kasei, boron nitride and C9-15 fluoroalcol phosphates and triethoxy caprylylsilane: FOTS-52 Boron Nitride available from Daito Kasei, mica and titanium dioxide and methicone: SI Sericite TI-2 available from Miyoshi Kasei, mica and titanium dioxide and methicone: SI Mica TI-2 available from Miyoshi Kasei, talc and titanium dioxide and methicone: SI Talc TI-2 available from Miyoshi Kasei, lauroyl lysine: AMIHOPE LL available from Ajinomoto, synthetic fluorphlogopite and methicone: PDM-5L(S)/PDM-10L(S)/PDM-20L(S)/PDM-40L(S) available from Topy Industries.

### Adhesive Agents

The compositions of the present invention can comprise from about 0.1% to about 10%, preferably from about 0.1% to about 2% of an adhesive agent. The species and levels of the adhesive agents are selected to provide, for example, a more flexible, longer-lasting benefit to composition, and/or better compatibility with other skin care or cosmetic formulations.

Examples of suitable adhesive agents include polyurethanes, including Polyderm PE-PA, available from Alzo International Inc.; co-polymerized amido ester compounds, including Polyderm PPG-17, available from Alzo International Inc.; and Acrylic Latex Dispersions.

### Skin Active Agents

The compositions of the present invention may comprise a skin active agent which provides a particular skin care benefit characteristic of the usage of the skin care product. Herein, skin care benefit may include benefits related to appearance or make-up of the skin. The skin care active can provide acute (immediate and short lived) benefits, or chronic (long term and longer lasting) benefits.

The term “skin active agent” as used herein, means an active ingredient which provides a cosmetic and/or therapeutic effect to the area of application on the skin. The skin active agents useful herein include skin lightening agents, anti-acne agents, emollients, non-steroidal anti-inflammatory agents, topical anaesthetics, artificial tanning agents, anti-microbial and anti-fungal actives, skin soothing agents, sun screening agents, skin barrier repair agents, anti-wrinkle agents, anti-skin atrophy actives, lipids, sebum inhibitors, sebum inhibitors, skin sensates, protease inhibitors, anti-itch agents, hair growth inhibitors, desquamation enzyme enhancers, anti-glycation agents, and mixtures thereof. When included, the present composition comprises from about 0.001% to about 20%, preferably from about 0.1% to about 10% of at least one skin active agent.

The type and amount of skin active agents are selected so that the inclusion of a specific agent does not affect the stability of the composition. For example, hydrophilic agents may be incorporated in an amount soluble in the aqueous phase, while lipophilic agents may be incorporated in an amount soluble in the oil phase.

Other skin active agents purported to exhibit expression-line relaxing benefits for use in the present invention include, but are not limited to, Lavandox available from Barnet Products Corporation; Thallasine 2, available from BiotechMarine; Argireline NP, available from Lipotec; Gatuline In-Tense and Gatuline Expression, available from Gattefosse; Myoxinol LS 9736 from BASF Chemical Company, Syn-ake, available from DSM Nutritional Products, Inc.; and Instensyl®, available from Silab, Inc; Sesaflash™, available from Seppic Inc.

Skin lightening agents useful herein refer to active ingredients that improve hyperpigmentation as compared to pre-treatment. Useful skin lightening agents herein include ascorbic acid compounds, vitamin B3 compounds, azelaic acid, butyl hydroxyanisole, gallic acid and its derivatives, glycyrrhizinic acid, hydroquinone, kojic acid, arbutin, mulberry extract, and mixtures thereof. Use of combinations of skin lightening agents is believed to be advantageous in that they may provide skin lightening benefit through different mechanisms.

Ascorbic acid compounds useful herein include ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein include, for example, esters of ascorbic acid, and ester salts of ascorbic acid. Particularly preferred ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-o-D-glucopyranosyl-L-ascorbic acid available from Hayashibara and sodium L-ascorbyl phosphate with tradename STAY C available from Roche.

Vitamin B3 compounds useful herein include, for example, those having the formula:

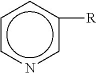

wherein R is —CONH2 (e.g., niacinamide) or —CH2OH (e.g., nicotinyl alcohol); derivatives thereof; and salts thereof. Exemplary derivatives of the foregoing vitamin B3 compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B3 compounds are niacinamide and tocopherol nicotinate, and more preferred is niacinamide. In a preferred embodiment, the vitamin B3 compound contains a limited amount of the salt form and is more preferably substantially free of salts of a vitamin B3 compound. Preferably the vitamin B3 compound contains less than about 50% of such salt, and is more preferably essentially free of the salt form. Commercially available vitamin B3 compounds that are highly useful herein include niacinamide USP available from Reilly.

Other hydrophobic skin lightening agents useful herein include ascorbic acid derivatives such as ascorbyl tetraisopalmitate (for example, VC-IP available from Nikko Chemical), ascorbyl palmitate (for example available from Roche Vitamins), ascorbyl dipalmitate (for example, NIKKOL CP available from Nikko Chemical); undecylenoyl phenyl alanine (for example, SEPIWHITE MSH available from Seppic); octadecenedioic acid (for example, ARLATONE DIOIC DCA available from Uniquema); oenothera biennis sead extract, and pyrus malus (apple) fruit extract, Water and Myritol 318 and butylene glycol and tocopherol and sscorbil tetraisopalmitate and Paraben and Carbopol 980 and DNA/SMARTVECTOR UV available from COLETICA, magnesium ascorbyl phosphate in hyaluronic filling sphere available from COLETICA, and mixtures thereof.

Other skin active agents useful herein include those selected from the group consisting of N-acetyl D-glucosamine, panthenol (e.g., DL panthenol available from Alps Pharmaceutical Inc.), tocopheryl nicotinate, benzoyl peroxide, 3-hydroxy benzoic acid, flavonoids (e.g., flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate: DL-α-tocopheryl acetate available from Eisai), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof. In a preferred example, the content level of a skin active agent is from about 0.001% to about 20%, more preferably from about 0.1% to about 10%

### Other Optional Components

The compositions hereof may further contain additional components such as those conventionally used in topical products, e.g., for providing aesthetic or functional benefit to the composition or skin, such as sensory benefits relating to appearance, smell, or feel, therapeutic benefits, or prophylactic benefits (it is to be understood that the above-described required materials may themselves provide such benefits).

These components may include, but are not limited to, materials purported to smooth, firm or lift sagging or wrinkled skin including: Quicklift, available from BASF Chemical Company; Syntran PC5100, available from Interpolymer Corporation; Glycolift, available from Solabia USA Inc.; Alguard, available from Frutarom; Easyliance, from Soliance; and Phytodermina Lifting code 9002, available from Istituto Ricerche Applicate.

The CTFA Cosmetic Ingredient Handbook, Second Edition (1992) describes a wide variety of nonlimiting cosmetic and pharmaceutical ingredients commonly used in the industry, which are suitable for use in the topical compositions of the present invention. Such other materials may be dissolved or dispersed in the composition, depending on the relative solubilities of the components of the composition.

### UV Protection Powder

UV protection powder provides UV protection benefit in the composition. UV protection powder has a particle size of less than 100 nm, which size provide very little coverage effect to the skin. The composition of each layer of the present invention may comprise from about 0% to about 20%, preferably from about 0.1% to about 10% of a UV protection powder, such as micronized titanium dioxide and micronized zinc oxide. The powder included in the pigment component herein is typically hydrophobic in nature, or hydrophobically treated.

Commercially available UV protection powder is titanium dioxide and methicone SI-TTO-S-3Z available from Miyoshi Kasei, titanium dioxide and dimethicone and aluminum hydroxide and stearic acid: SAST-UFTR-Z available from Miyoshi Kasei, Zinc oxide: Finex series available from Sakai Chemical Industry.

### UV Absorbing Agent

The compositions of the present invention may comprise a safe and effective amount of a UV absorbing agent. A wide variety of conventional UV protecting agent are suitable for use herein, such as those described in U.S. Pat. No. 5,087,445, Haffey et al, issued Feb. 11, 1992; U.S. Pat. No. 5,073,372, Turner et al, issued Dec. 17, 1991; U.S. Pat. No. 5,073,371, Turner et al., issued Dec. 17, 1991; and Segarin, et al, at Chapter VIII, pages 189 et seq., of Cosmetics Science and Technology (1972). When included, the present composition comprises from about 0.5% to about 20%, preferably from about 1% to about 15% of a UV absorbing agent.

UV absorbing agent useful herein includes, for example, 2-ethylhexyl-p-methoxycinnamate (commercially available as PARS OL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, Octocrylene, Avobenzone (commercially available as Parsol 1789), and mixtures thereof.

### Thickener

Useful for the present invention is a thickener. Thickeners can be used for solidifying solid water-in-oil form compositions of the present invention. When used, the thickener is kept to about 15% or less of the composition. The thickeners useful herein are selected from the group consisting of fatty compounds, gelling agents, inorganic thickeners and mixtures thereof. The amount and type of thickeners are selected according to the desired viscosity and characteristics of the product. These characteristics may include a synergistic effect between the thickener and the film forming ingredients, thereby enhancing product/film adhesion, contraction, or flexibility, while decreasing whiteness.

Thickening agents which can be used in the present invention include, but are not limited to, cross-linked polyacrylates such as CarbopolTM (Goodrich); polyacrylate copolymers such as SepiMAX ZEN (Seppic, Inc.); modified acrylate copolymers such as Sepiplus S (Seppic, Inc.) polymeric carboxylates including modified and unmodified starches, polysaccharide gums such as xanthan gum (e.g. CP Kelco's Keltrol CGT and Keltrol T630, Jungbunzlauer's Xanthan Gum), dehydroxanthan gum (e.g. Amaze XT from AkzoNobel), gallactomanan ((Solagum Tara from Seppic), and cellulose derivatives (e.g. Natrosol 250). Gums may also include, but are not limited to, crosslinked-xanthan gum, hydroxypropyl xanthan gum, undecylenoyl xanthan gum, deacetylated xanthan gum, guar gum, cellulose gum, carrageenan, hydroxylpropyl methyl cellulose, and sodium carboxymethyl chitin.

Polymers useful herein include swellable, lightly to moderately crosslinked polyvinyl pyrrolidones (PVP) such as ACP-1120 (International Specialty Products), acrylate copolymers/crosspolymers/blends such as acrylate/steareth-20 itaconate copolymer (Structure 2001 from AkzoNobel), acrylates/C10-30 alkyl acrylates copolymer (Amaze XT from AkzoNobel), acrylic acid/VP crosspolymer (Ultrathix P100 from International Specialty Products).

Fatty compounds useful herein include stearic acid, palmitic acid, stearyl alcohol, cetyl alcohol, behenyl alcohol, stearic acid, palmitic acid, the polyethylene glycol ether of stearyl alcohol or cetyl alcohol having an average of about 1 to about 5 ethylene oxide units, and mixtures thereof. Preferred fatty compounds are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof.

The gelling agent useful as thickeners of the present invention include esters and amides of fatty acid gellants, hydroxy acids, hydroxy fatty acids, other amide gellants, and crystalline gellants. N-acyl amino acid amides useful herein are prepared from glutamic acid, lysine, glutamine, aspartic acid and mixtures thereof.

### Silicone Elastomer

Soft focus silicone elastomer is crosslinked siloxane elastomer which is particularly effective in providing soft focus effect to the skin In other words, when incorporated in a cosmetic product a defined amount of silicone elastomer, the silicone elastomer can provide natural finish yet having good coverage for minimizing the appearance of skin troubles. Specifically, silicone elastomer has lower matte level compared with other silicone oil. Matte level is a parameter reflecting soft focus effect, i.e. natural finish of a cosmetic material. The lower the matte level is, the better natural finish the material can provide. Matte level of silicone elastomer used in the present application is less than about 40. Matte level can be measured by the PG-1M gloss meter (Incidence angle/Reflection angle:)60/60° made by Nihon Denshoku Kogyo. Commercially available silicone elastomer useful in the present application includes a silicone elastomer having the tradename KSG-16 available from Shinetsu, which has a matte level of about 37.

Silicone elastomer suitable for use herein can be emulsifying or non-emulsifying crosslinked siloxane elastomer or mixtures thereof. The term “non-emulsifying” as used herein, defines crosslinked organopolysiloxane elastomer from which polyoxyalkylene units are absent. The term “emulsifying” as used herein, means crosslinked organopolysiloxane elastomer having at least one polyoxyalkylene (e.g., polyoxyethylene or polyoxypropylene) unit. Non-emulsifying elastomer useful in the present invention is formed via crosslinking organohydroenpolysiloxane with an alpha, omega-diene. Emulsifying elastomer herein includes polyoxyalkylene modified elastomer formed via cros slinking from organohydrogenpolysiloxane with polyoxyalkylene diene or organohydrogenpolysiloxane containing at least one polyether group crosslinked with an alpha, omega-diene. Emulsifying crosslinked organopolysiloxane elastomer can notably be chosen from the crosslinked polymer described in U.S. Pat. Nos. 5,412,004, 5,837,793, and 5,811,487. In addition, an emulsifying elastomer comprised of dimethicone copolyol crosspolymer (and dimethicone) is available from Shin Etsu under the tradename KSG-21.

Non-emulsifying elastomer is dimethicone/vinyl dimethicone crosspolymer. Such dimethicone/vinyl dimethicone crosspolymer is supplied by a variety of suppliers including Dow Coming (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomer). Cross-linked organopolysiloxane elastomer useful in the present invention and processes for making them are further described in U.S. Pat. Nos. 4,970,252, 5,760,116, and 5,654,362. Additional crosslinked organopolysiloxane elastomer useful in the present invention is disclosed in Japanese Patent Application JP 61-18708, assigned to Pola Kasei Kogyo KK. Commercially available elastomer preferred for use herein is Dow Coming's 9040 silicone elastomer blend, Shin Etsu's KSG-21, and mixtures thereof.

Similar to the radiant powder, when formulated with coverage titanium dioxide in a single layer, the content level of a silicone elastomer shall be as high as 10% to achieve noticeable natural look effect. However, in the present invention, by formulating a silicone elastomer mainly in the second layer and coverage titanium dioxide in the first layer, and providing the first and second layers in a manner such that they can be simultaneously applied on the skin, the skin care product of the present invention can provide satisfied natural look effect with lower level of silicone elastomer. As a result, the cost of the product can be controlled while also providing more flexibility in product formulation. In a preferred example, the content level of silicone elastomer in the second layer is from about 1% to about 20%, preferably from about 2% to about 15%. When calculated based on the total weight of the first layer and the second layer, the preferred content level of silicone elastomer is from about 0.5% to about 8%, more preferably from about 1% to about 5%.

### Oil Absorbing Powder

Oil absorbing powder is a pigment that is particularly effective in absorbing oil, and thereby can be included in the present composition for absorbing excessive sebum from the skin Specifically, the oil absorbing powder herein has an oil absorbency of at least about 100 ml/100 g, preferably at least about 200 ml/100 g. Oil absorbency is a unit well known to the artisan, and which can be measured via: JIS K5101 No.21 “Test Method for Oil Absorbency Level”.

Oil absorbing powder useful herein includes spherical silica, and methyl methacrylate copolymer. Commercially available spherical oil absorbing pigments useful herein include spherical silica with tradename SI-SILDEX H-52 available from Miyoshi Kasei, Inc. having an oil absorbency of more than 200 ml/100 g, vinyl dimethicone/methicone silsesquioxane crosspolymer with tradename KSP-100 and KSP-101 available from ShinEtsu Chemical having an oil absorbency of more than 200 ml/100 g, and methyl methacrylate copolymer with tradename SA-GMP-0820 available from GANZ Chemical and surface treated by Miyoshi Kasei, Inc. having an oil absorbency of more than 100 ml/100 g. Typically, inclusion of oil absorbing powder for oil shine control may provide a composition with unfavorable spreadability performance. However, in the present invention, by including oil absorbing powder mainly in the second layer, the unfavorable spreadability performance can be improved. In a preferred example, the content level of an oil absorbing powder in the second layer is from about 1% to about 10%, more preferably from about 3% to about 5%.

### Methods of Treatment

Various methods of treatment, application, regulation, or improvement may utilize the aforementioned compositions. Application of the present compositions can occur on any skin surface of the body. Skin surfaces of the most concern tend to be those not typically covered by clothing such as facial skin surfaces, hand and arm skin surfaces, foot and leg skin surfaces, and neck and chest skin surfaces (e.g., décolletage). In particular, application may be on a facial skin surface including the forehead, perioral, chin, periorbital, nose, and/or cheek skin surfaces.

Many regimens exist for the application of the composition to the skin. The composition may be applied at least once a day, twice a day, or on a more frequent daily basis, during a treatment period. When applied twice daily, the first and second applications are separated by at least 1 to about 12 hours. Typically, the composition may be applied in the morning and/or in the evening.

The step of applying the composition to the skin may be done by localized application to an area that contains wrinkles In reference to application of the composition, the term “localized”, “local”, or “locally” mean that the composition is delivered the targeted area (such as an area of skin containing wrinkles) while minimizing delivery to skin surface not requiring treatment. The composition may be applied and lightly massaged into the skin. It is recognized that localized application allows for a reasonable amount of the composition to be applied to areas adjacent the wrinkles to be treated (i.e., the composition is unlikely to be applied or to remain within the boundary of the wrinkles without some spreading). The form of the composition or the dermatologically acceptable carrier should be selected to facilitate localized application. While certain embodiments of the present invention contemplate applying a composition locally to a wrinkled area, it will be appreciated that compositions of the present invention can be applied more generally or broadly to one or more facial skin surfaces to reduce the appearance of wrinkles within those facial skin regions. Likewise, the compositions can be applied as a continuous film, or in patterns. Striations, patterned spots or random application of the compositions may be desirable. Applicators, as described below, may be beneficial assisting in patterned deposition.

According to a particular method, the compositions may be applied to skin regions where a desired lifting effect is desired. For example, the composition may be applied to the hair-line, temples, jaw-line, and other peripheral facial regions in order to apply a lifting effect to other facial regions. This method utilizes a lifting effect at the periphery of the face to reduce the appearance of wrinkles at, for example, the eye region, smile lines around the mouth, under-eye wrinkles, and to smooth wrinkles around cheek areas. According to this method, the composition may be applied to the periphery of the face without applying the composition directly to target wrinkles.

### Applicators

In some embodiments, the composition may be delivered by a variety of applicators appropriate for localized and general application. By way of example, a suitable applicator may be a dropper and bottle that contains the composition. A pen-like wand with a housing that may contain the composition can also be used. The wand may comprise a handle, a stem, and an applicator head. The applicator head may comprise fibers, foam, cotton, a roller ball or any other suitable material that may releasably hold the composition.

A simple cotton swab can apply the composition locally to the wrinkled area. Other suitable applicators include SH-0127 pen applicator available from Shya Hsin Plastic Works, Inc., Taiwan and either the Xpress Tip or liquid filled swab available from SwabPlus, Inc., China. The applicator may be configured to easily apply the composition to wrinkled areas having an approximate diameter between about 2 mm and about 20 mm and allowing for a dosed amount of the composition of between about 0.01 to about 2 mg/cm2 or between about 0.1 to about 1 mg/cm2. Thickness of the applied film can be measured or calculated based on the application are and application dose given directly above.

In another embodiment, the applicator may be in the form of a pretreated tape. The tape may be treated or impregnated with the composition herein, then applied to skin via any suitable tape-dispensing mechanism. In a particularly preferred embodiment, the tape may comprise a dissolvable material, such as a water-soluble polyvinyl alcohol (PVA) film. Such a material allows the user to apply the pretreated tape without having to remove the tape backing upon application since the backing would eventually dissolve, leaving only the composition on the treated site.

While some methods described herein contemplate applying the compositions of the present invention with an applicator, it will be appreciated that applicators are not required and the compositions of the present invention can also be applied directly by using one's finger or in other conventional manners.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention, as are ranges based thereon. Where a combination is disclosed, each subcombination of the elements of that combination is also specifically disclosed and is within the scope of the invention. Conversely, where different elements or groups of elements are disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic composition comprising:
a) from about 20 wt. % to about 80 wt. % of a tensor agent; and
b) a dermatologically acceptable carrier;
c) wherein said tensor agent is a polyester/polyurethane block copolymer, and wherein said tensor agent is capable of forming an anisotropic microstructure on skin.

2. A cosmetic composition according to claim 1, wherein said composition comprises less than about 10% of a skin conditioning agent.

3. A cosmetic composition according to claim 2, wherein said conditioning agent is selected from humectants and emollients.

4. A cosmetic composition according to claim 3, wherein said conditioning agent is at least one humectants, said humectants being present at a level of from about 2 wt. % to about 6 wt %.

5. A cosmetic composition according to claim 1, wherein said composition comprises less than about 5% of a plasticizer.

6. A cosmetic composition according to claim 1, wherein said carrier is present at a level of from about 20% to about 80% of the composition.

7. A cosmetic composition according to claim 1 further comprising a pigment powder.

8. A cosmetic composition according to claim 6, wherein said pigment powder is an inorganic powder.

9. A cosmetic composition according to claim 6, wherein said pigment powder is an organic powder.

10. A cosmetic composition according to claim 1, further comprising an adhesive agent.

11. A cosmetic composition according to claim 1, further comprising one or more skin actives selected from the group consisting of skin lightening agents, anti-acne agents, non-steroidal anti-inflammatory agents, topical anaesthetics, artificial tanning agents, anti-microbial actives, anti-fungal actives, skin soothing agents, sun screening agents, skin barrier repair agents, anti-wrinkle agents, anti-skin atrophy actives, lipids, sebum inhibitors, sebum inhibitors, skin sensates, protease inhibitors, anti-itch agents, hair growth inhibitors, desquamation enzyme enhancers, anti-glycation agents, and mixtures thereof.

12. A cosmetic composition according to claim 1, further comprising a thickening agent.

13. A cosmetic composition according to claim 1, further comprising a silicone elastomer.

14. A cosmetic composition according to claim 1, further comprising an oil absorbing powder.
